(19) United States
(12) Patent Application Publication
(10) Pub. No.: US 2007/0135788 A1
(43) Pub. Date: Jun. 14, 2007

(54) Pants liner

(76) Inventors: Emmanuelle Damay, Erlangen (DE); Franz Aschenbrenner, Kastl (DE); Peter Ludwig Huebner, Winkelhaid (DE); Herbert Ellsworth Grube, Greenville, WI (US)

Correspondence Address: KIMBERLY-CLARK WORLDWIDE, INC., 401 NORTH LAKE STREET, NEENAH, WI, 54956, US

(21) Appl. No.: 11/302,032

(22) Filed: Dec. 12, 2005

(51) Int. Cl. A61F 13/15

(52) U.S. Cl. 604/385.01; 604/385.201

(57) ABSTRACT

A personal care article (20) has a longitudinal-direction (22); a relatively shorter, lateral cross-direction (24); a first end-edge (74); a longitudinally-opposed second end-edge (78); a first side-edge (62); and a laterally-opposed second side-edge (64). The article includes a liquid-permeable topsheet layer (26), and a backsheet layer (28) operatively connected in facing relation with the topsheet layer. A first end-notch (80) is formed to extend inward from the first end-edge (74), and a second end-notch (82) is formed to extend inward from the second end-edge (78). In a particular aspect, at least a first, end-line of bending weakness (88) extends from an inboard, apex end region of the first end-notch (80). In other aspects, a first side-notch (66) can be formed to extend inward from the first side-edge (62), and a second side-notch (70) can be formed to extend inward from the second side-edge (64).

### FIELD OF THE INVENTION

The present invention relates to a personal care article. More particularly, the present invention pertains to a personal care absorbent article, such as an absorbent feminine care or adult care pad. The personal care article can be operatively secured to a selected outer-garment of a wearer.

### BACKGROUND OF THE INVENTION

Absorbent products intended to absorb discharged body fluids are well known in the art. Such absorbent products generally comprise a fibrous mass or other absorbent body which can absorb and hold the body fluids. Similarly, it is well known that feminine care articles have been employed to absorb and hold liquids, such as urine and/or menses. The absorbent articles have included various systems of liquid-handling layers, such as intake layers, distribution layers, retention layers and the like. Typically, a garment-attachment adhesive has been employed to secure the article to a wearer's undergarment. Additionally, the absorbent articles have included wing portions which can help to hold the article in place at a selected location in the undergarment. Various fasteners have been employed to secure the wing portions in a desired configuration during ordinary use. The fasteners have included adhesive fasteners as well as mechanical fasteners, and the mechanical fasteners have included conventional, hook-and-loop fasteners. Individual absorbent articles have been folded or rolled to reduce the size of the articles for storage prior to use, and each article has been enclosed in a corresponding, individual storage pouch or other container.

In other arrangements, absorbent articles have been configured for placement directly against the crotch region of a user's outer garment, such as a user's outer pants garment. Typically, an adhesive has been employed to secure the,article to a wearer's outer garment. Where the absorbent article has been configured for direct placement against the user's outer garment, the user has typically decided to not wear an undergarment, such as a panty or underpants.

Conventional absorbent articles that have been configured for use without an undergarment, however, have not readily conformed to the crotch region of the wearer's outer garment, and have not been sufficiently able to maintain a desired positioning in the outer garment. As a result, there has been a continued need for an improved article that can be more effectively maintain the desired positioning in the outer garment, while also providing a sufficient level of discretion and leakage protection.

### BRIEF DESCRIPTION OF THE INVENTION

Generally stated, the present invention provides a personal care article having a longitudinal-direction; a relatively shorter, lateral cross-direction; a first end-edge; a longitudinally-opposed second end-edge; a first side-edge; and a laterally-opposed second side-edge. The article includes a liquid-permeable topsheet layer, and a backsheet layer that is operatively connected in facing relation with the topsheet layer. A first end-notch is formed to extend inward from the first end-edge, and second end-notch is formed to extend inward from the second end-edge. In a particular aspect, at least a first end-line of bending weakness extends from an inboard, apex end region of the first end-notch. In desired aspects, a first side-notch can be formed to extend inward from the first side-edge, and a second side-notch can be formed to extend inward from the second side-edge.

In a particular feature, the article can include a second end-line of bending weakness which extends from an inboard, apex end region of the second end-notch. In other features, a first, complementary, end-line of bending weakness can extend from the inboard end region of the first end-notch; and a second, complementary, end-line of bending weakness can extend from the inboard end region of the second end-notch.

By incorporating its various features and configurations, the article of the invention can better conform to the shape of the crotch region of the wearer's outer garment, and can more effectively maintain a desired positioning within the outer garment. Additionally, the article can more effectively provide the desired levels of discretion and leakage protection.

### BRIEF DESCRIPTION OF THE DRAWINGS

The various features, aspects and advantages of the present invention will become better understood with reference to the following description, appended claims and accompanying drawings where:

FIG. 1 shows a representative, partially cut-away, top, plan view of a bodyside of a personal care article.

FIG. 2 shows a partially cut-away, bottom, plan view of a garment-side of a representative personal care article.

FIG. 3 shows an expanded, schematic view of a representative, longitudinal cross-section of a personal care article.

FIG. 4 shows a schematic view of a representative article disposed in a cooperating pants garment that is illustrated in phantom lines.

FIG. 5 shows a representative graph produced from typical data generated by a trapezoid tear test.

### DETAILED DESCRIPTION OF THE INVENTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

By the terms “particle,” “particles,” “particulate,” “particulates” and the like, it is meant that the material is generally in the form of discrete units. The units can comprise granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

As used herein, the term “nonwoven” refers to a fabric web that has a structure of individual fibers or filaments which are interlaid, but not in an identifiable repeating manner.

As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

“Coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein, the phrase “complex liquid” describes a liquid generally characterized as being a viscoelastic liquid comprising multiple components having inhomogeneous physical and/or chemical properties. It is the inhomogeneous properties of the multiple components that challenge the efficacy of an absorbent or adsorbent material in the handling of complex liquids. In contrast with complex liquids, simple liquids, such as, for example, urine, physiological saline, water, and the like, are generally characterized as being relatively low-viscosity and comprising one or more components having homogeneous physical and/or chemical properties. As a result of having homogeneous properties, the one or more components of simple liquids behave substantially similarly during absorption or adsorption, although some components may be absorbed or adsorbed more readily than others.

Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells.

As used herein, the term “hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials can be provided by a Cahn SFA-222 Surface Force Analyzer System, or a substantially equivalent system. When measured with this system, fibers having contact angles less than 90° are designated “wettable” or hydrophilic, while fibers having contact angles equal to or greater than to 90° are designated “nonwettable” or hydrophobic. When comparing materials, a material that forms a relatively larger contact angle with water is relatively less hydrophilic than a material that forms a smaller contact angle with water.

As used herein, the phrase “absorbent article” refers to devices which absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, tampons, interlabial devices and the like), infant diapers, children's training pants, adult incontinence products and the like; as well as absorbent wipes and covering mats.

Disposable absorbent articles such as, for example, many of the feminine care absorbent products, can include a liquid pervious topsheet, an operatively liquid-impervious backsheet joined to the topsheet, and an absorbent core positioned and held between the topsheet and the backsheet. The topsheet is operatively permeable to the liquids that are intended to be held or stored by the absorbent article, and the backsheet may be substantially impermeable or otherwise operatively impermeable to the intended liquids. The absorbent article may also include other components, such as liquid wicking layers, liquid intake layers, liquid distribution layers, transfer layers, barrier layers, and the like, as well as combinations thereof. Disposable absorbent articles and the components thereof can operate to provide a body-facing surface and a garment-facing surface. As used herein, the body-facing or bodyside surface means that surface of the article or component which is intended to be disposed toward or placed adjacent to the body of the wearer during ordinary use, while the outward, outward-facing or garment-side surface is on the opposite side, and is intended to be disposed to face away from the wearer's body during ordinary use. Such outward surface may be arranged to face toward or placed adjacent to the wearer's undergarments when the absorbent article is worn.

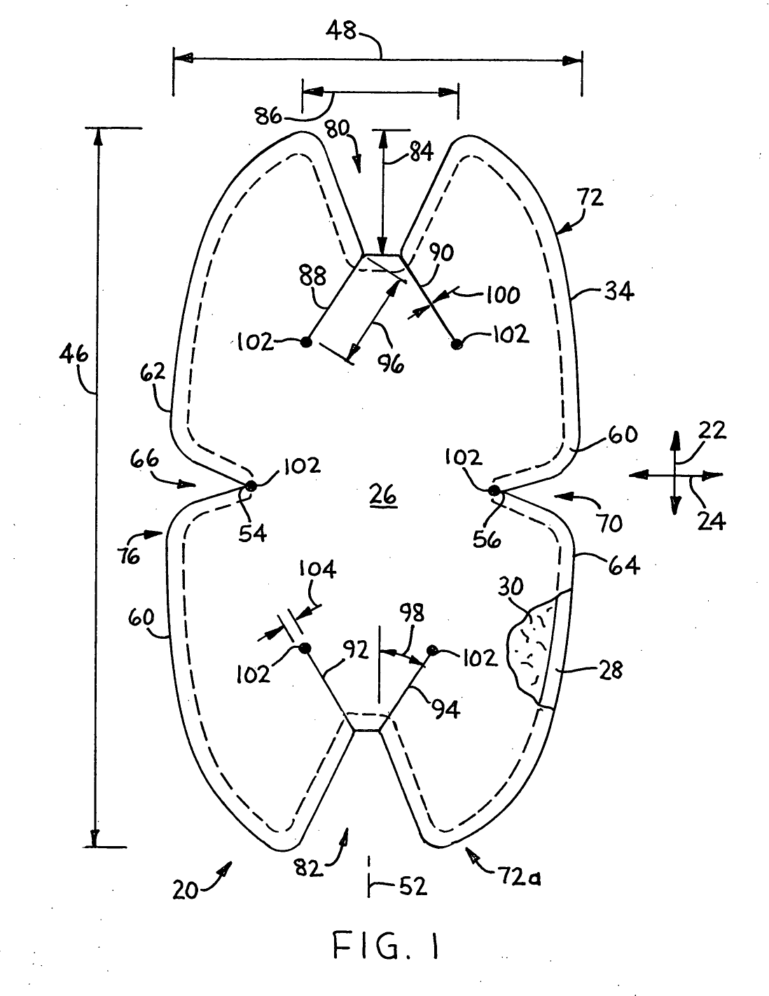

FIGS. 1 through 4, illustrate an example of a suitable personal care article 20, such as the representatively shown adult care article, which is configured to incorporate the present invention. The adult care article can, for example, be an adult incontinence product, or a feminine care pad or napkin. As representatively shown, the personal care article 20 has a longitudinal-direction 22; a relatively shorter, lateral cross-direction 24; and a thickness-direction which extends perpendicular to both the longitudinal-direction and cross-direction. The article also has a first end-edge 74; a longitudinally-opposed second end-edge 78; a first side-edge 62; and a laterally-opposed second side-edge 64. The article includes a liquid-permeable topsheet layer 26, and a backsheet layer 28 operatively connected in facing relation with the topsheet layer. Optionally, the article may include an absorbent body 30 which is operatively sandwiched and assembled between the topsheet and backsheet layers. A first end-notch 80 is formed to extend inward from the first end-edge 74, and can be provided with a first end-notch length 84 and a first end-notch width 86. A second end-notch 82 is formed to extend inward from the second end-edge 78, and can be provided with a second end-notch length and a second end-notch width. A particular aspect of the article can include at least a first end-line of bending weakness (e.g. end-slit 88) which extends from an inboard, apex end region of the first end-notch 80. In other aspects, a first side-notch 66 can be formed to extend inward from the first side-edge 62, and a second side-notch 70 can be formed to extend inward from the second side-edge 64. The first side-notch 66 can be formed to extend inward from the first side-edge 62 by a first side-notch length, and can be formed with a first side-notch width. Additionally, the second side-notch 70 can be formed to extend inward from the second side-edge 64 by a second side-notch length, and can be formed with a second side-notch width.

In still another aspect, the article can include a second end-line of bending weakness (e.g. end-slit 92) can be configured to extend from an inboard, apex end region of the second end-notch 82. In further aspects, a first, complementary, end-line of bending weakness (e.g. a first complementary end-slit 90) can extend from the inboard end region of the first end-notch 80; and a second, complementary, end-line of bending weakness (e.g. a second complementary end-slit 94) can extend from the inboard end region of the second end-notch 82.

By incorporating its various features and configurations, alone or in desired combinations, the invention can provide a more effective personal care article. The article can better conform to the shape of the crotch region of the wearer's outer garment, and can more effectively maintain a desired positioning within the outer garment. Additionally, the article can more effectively provide the desired levels of discretion and leakage protection.

The overall personal care article 20 can be configured to be extensible, substantially non-extensible, elastomeric or substantially non-elastomeric, as desired. Additionally, the individual components of the article (e.g. topsheet, backsheet, and/or absorbent) can be configured to be extensible, substantially non-extensible, elastomeric or substantially non-elastomeric, as desired. The extensibility and/or elastomeric stretchability of the article or components can, for example, help provide improved comfort and improved fit into a wearer's pants.

With reference to FIGS. 1 through 3, the topsheet 26 may include a layer constructed of any operative material, and may be a composite material. The topsheet may be extensible, elastomeric, substantially non-extensible or substantially non-elastomeric. For example, the topsheet layer can include a woven fabric, a nonwoven fabric, a polymer film, a film-fabric laminate or the like, as well as combinations thereof. Examples of a nonwoven fabric include spunbond fabric, meltblown fabric, coform fabric, coform fabric that includes elastomeric fibers, airlaid fibrous web, airlaid fibrous web that includes elastomeric fibers, a carded web, a bonded-carded-web, a bicomponent spunbond fabric or the like as well as combinations thereof. For example, the topsheet layer can include a woven fabric, a nonwoven fabric, a polymeric film that has been configured to be operatively liquid-permeable, or the like, as well as combinations thereof. Other examples of suitable materials for constructing the topsheet layer can include rayon, cotton, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A particular example of a suitable topsheet layer material can include a bonded-carded-web composed of polypropylene and polyethylene, such as has been used as a topsheet stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. In another example, the article can include a topsheet 26 composed of a soft, bonded-carded web having a basis weight of about 22 g/m². The topsheet can be thermally bonded to an airlaid absorbent having a basis weight of about 63 g/m². A further example of the topsheet 26 can be composed of a bilayer spunlace material having a basis weight of about 80 g/m², wherein the top layer is made of polypropylene and viscose fiber, and the bottom layer is made of 100% viscose fibers. Other examples of suitable materials can include composite materials of a polymer and a nonwoven fabric material. The composite materials can be in the form of integral sheets which can be formed by the extrusion of a polymer onto a web of spunbond material.

In a desired arrangement, the topsheet layer 26 can be configured to be operatively liquid-permeable with regard to the liquids that the article is intended to absorb or otherwise handle. The operative liquid-permeability may, for example be provided by a plurality of pores, perforations, apertures or other openings, as well as combinations thereof, that are present or formed in the topsheet layer. The apertures or other openings can help increase the rate at which bodily liquids can move through the thickness of the topsheet layer and penetrate into the other components of the article (e.g. into the absorbent structure 30). The selected arrangement of liquid-permeability is desirably present at least on an operative portion of the topsheet layer that is appointed for placement on the body-side of the article. The topsheet layer 26 can provide comfort and conformability, and can function to direct bodily exudates away from the body and toward the absorbent structure 30. In a desired feature, the topsheet layer 26 can be configured to retain little or no liquid in its structure, and can be configured to provide a relatively comfortable and non-irritating surface next to the body-tissues of a female wearer. The topsheet layer 26 can be constructed of any material which is also easily penetrated by bodily fluids that contact the surface of the topsheet layer.

The topsheet 26 can also have at least a portion of its bodyside surface treated with a surfactant to render the topsheet more hydrophilic. The surfactant can permit arriving bodily liquids to more readily penetrate the topsheet layer. The surfactant may also diminish the likelihood that the arriving bodily fluids, such as menstrual fluid, will flow off the topsheet layer rather than penetrate through the topsheet layer into other components of the article (e.g. into the absorbent body structure). In a particular configuration, the surfactant can be substantially evenly distributed across at least a portion of the upper, bodyside surface of the topsheet 26 that overlays the upper, bodyside surface of the absorbent.

The topsheet layer can optionally be treated with other treatment materials. The treatment materials can, for example, include waxed lotions, and can include agents such as chamomile, aloe vera, avocado oil, green tea, or the like, as well as combinations thereof. Other treatment materials can include scents, anti bacterial agents, control agents or the like, as well as combinations thereof. In particular arrangements, the treatment materials can be incorporated into microcapsules.

Where the article includes the absorbent body 30, the topsheet 26 may be maintained in secured relation with the absorbent structure by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the topsheet, or fusing at least portions of the adjacent surface of the topsheet to portions of the adjacent surface of the absorbent.

The topsheet 26 typically extends over the upper, bodyside surface of the absorbent structure, but can optionally extend around the article to partially or entirely, surround or enclose the absorbent structure. Alternatively, the topsheet 26 and the backsheet 28 can have peripheral margins which extend outwardly beyond the terminal, peripheral edges of the absorbent structure 30, and the extending margins of the topsheet and backsheet can be joined together to partially or entirely, surround or enclose the absorbent structure.

The backsheet 28 can be operatively connected to the topsheet layer 26 using any suitable technique or any direct or indirect configuration. The connection technique may, for example, include adhesive bonding, thermal bonding, sonic bonding, cohesive bonding, mechanical attachments or the like, as well as combinations thereof. The backsheet may include a layer constructed of any operative material, and may be extensible, elastomeric, substantially non-extensible or substantially non-elastomeric, as desired. Additionally, the backsheet layer may or may not have a desired level of liquid-permeability or a desired level of liquid-impermeability. In a particular configuration, the baffle or backsheet 28 can be configured to provide an operatively liquid-impermeable backsheet structure. The backsheet may, for example, include a polymeric film, a woven fabric, a nonwoven fabric or the like, as well as combinations or composites thereof. For example, the backsheet may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, have a printed message to the consumer, and/or may be at least partially colored. Desirably, the backsheet 28 can operatively permit a sufficient passage of air and moisture vapor out of the article, particularly out of an absorbent (e.g. storage or absorbent structure 30) while blocking the passage of bodily liquids. An example of a suitable backsheet material can include a breathable, microporous film, such as a HANJIN Breathable backsheet available from Hanjin Printing, Hanjin P&C Company Limited, a business having offices located in Sahvon-li.Jungan-mvu.Kongiu-City, Chung cheong nam-do, Republic of South Korea. The backsheet material is a breathable film, which is white in color, dimple embossed, and contains: 47.78% calcium carbonate, 2.22% TiO₂, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed cell polyolefin foam. For example, a closed cell polyethylene foam may be employed. Still another example of a backsheet material would be a material that is similar to a polyethylene film which is used on commercially sold KOTEX brand pantiliners, and is obtainable from Pliant Corporation, a business having offices located in Schaumburg, Ill., U.S.A.

The absorbent body 30 may be extensible, elastomeric, substantially non-extensible or substantially non-elastomeric, as desired. The structure of the absorbent body can also be operatively configured to provide a desired level of absorbency or storage capacity. More particularly, the absorbent body can be configured to hold a liquid, such as urine, menses, other complex liquid or the like, as well as combinations thereof. As representatively shown, the absorbent body can include a matrix of absorbent fibers and/or absorbent particulate material, and the absorbent fiber can include natural fiber (e.g. woodpulp fluff) and/or synthetic fiber. Optional arrangements of the absorbent body can comprise a coform fabric that includes elastomeric fibers, an airlaid fibrous web, an airlaid fibrous web that includes elastomeric fibers or the like, as well as combinations thereof. Additionally, the absorbent body may include one or more components that can modify menses or intermenstrual liquid.

The absorbent structure 30 may also include superabsorbent material. Superabsorbent materials suitable for use in the present invention are known to those skilled in the art, and may be in any operative form, such as particulate form. Generally stated, the superabsorbent material can be a water-swellable, generally water-insoluble, hydrogel-forming polymeric absorbent material, which is capable of absorbing at least about 20, desirably about 30, and possibly about 60 times or more its weight in physiological saline (e.g. saline with 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company and Stockhausen, Inc. The superabsorbent material may desirably be included in an appointed storage or retention portion of the absorbent system, and may optionally be employed in other components or portions of the absorbent article.

The amount of superabsorbent material in the absorbent body 30 can be up to about 75 wt % or more, as determined with respect to the total weight of material in the absorbent body. In particular aspects, the amount of superabsorbent material can be within the range of about 5-35 wt %, and can alternatively be within the range of about 8-20 wt % to provide desired performance. In desired configurations, the amount of superabsorbent can be about 15 wt %.

In particular configurations, the absorbent body 30 can be included in an adult care article, and can provide a composite, overall absorbent saturation capacity (saturated retention capacity) which is at least a minimum of about 2 grams of 0.9 wt % saline. The overall absorbent saturation capacity can alternatively be at least about 4 grams of saline to provide improved performance. In other aspects, the overall absorbent saturation capacity can be up to a maximum of about 12 grams of saline, or more, and can alternatively be up to about 10 grams of saline to provide improved effectiveness. In a desired arrangement, the composite, overall absorbent saturation capacity can be up to about 8 grams of saline.

In other configurations, the absorbent body 30 can be included in a feminine care article, and can provide a composite, overall absorbent saturation capacity which is at least a minimum of about 5.5 grams of menses simulant A. The overall absorbent saturation capacity can alternatively be at least about 40 grams of menses simulant A to provide improved performance. In other aspects, the overall absorbent saturation capacity can be up to a maximum of about 120 grams of menses simulant A, or more, and can alternatively be up to about 88 grams of menses simulant A to provide improved effectiveness. In a desired arrangement, the composite, overall absorbent saturation capacity can be about 60 grams of menses simulant A.

In particular configurations, the absorbent body 30 can be included in another feminine care article, and can provide a composite, overall absorbent retention capacity which is at least a minimum of about 5 grams of menses simulant A. The overall absorbent retention capacity can alternatively be at least about 10 grams of menses simulant A to provide improved performance. In other aspects, the overall absorbent retention capacity can be up to a maximum of about 34 grams of menses simulant A, or more, and can alternatively be up to about 20 grams of menses simulant A to provide improved effectiveness. In a desired arrangement, the composite, overall absorbent retention capacity can be about 14.5 grams of menses simulant A.

The menses simulant A is composed of swine blood diluted with swine plasma to provide a hematocrit level of 35% (by volume). A suitable device for determining the hematocrit level is a HEMATOSTAT-2 system, available from Separation Technology, Inc., a business having offices located in Altamonte Springs, Fla., U.S.A. A substantially equivalent system may alternatively be employed. Simulant A is typically used for absorbent capacity tests, where the viscoelastic properties that affect liquid movement have been found to be of little importance.

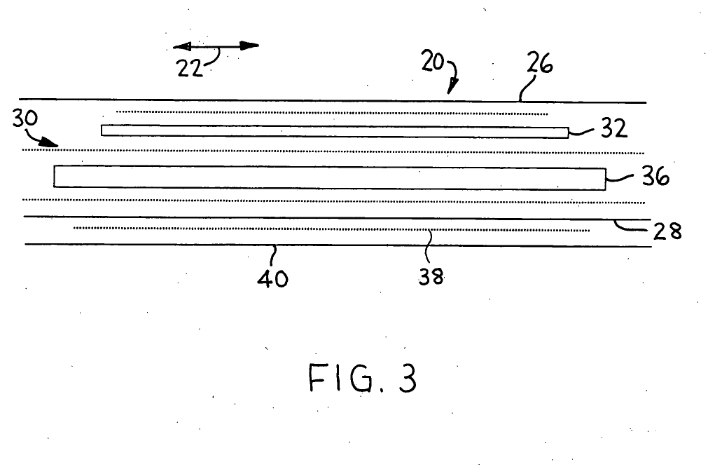

The absorbent body 30 can include any operative configuration, and may, for example, include a single unitary layer, or multiple layers, as desired. As representatively shown, the absorbent body 30 of the selected article can comprise a composite structure having a selected plurality of strata or layers. With reference to FIG. 3, for example, the absorbent composite can include an intake layer 32 and an absorbent shaping layer 36, as well as any other desired components, arranged in any operative combination. As representatively shown, the structure of the absorbent body can include an absorbent pad, shaping layer 36 which is positioned between the topsheet 26 and the backsheet 28, and can include an intake layer 32 which is positioned between the topsheet 26 and the shaping layer 36.

In a particular aspect, the article 20 can include a top, bodyside intake layer 32 which is sized and placed to more effectively operate in a target area of the absorbent body 30 where liquids are more likely to be introduced into the article. The material of the intake layer can be configured to provide desired liquid-intake properties, substantially without consideration for delivering shaping properties. For example, the configuration of the intake layer may or may not include properties that are configured to prevent bunching and twisting of the article, particularly the absorbent structure, during ordinary wear.

The intake layer can include material that is configured to quickly absorb and pull liquid away from the body. Accordingly, the intake layer 32 can provide the function of liquid intake and can also provide the functions of liquid distribution, spreading, temporary storage and liquid retention. The intake layer may include natural fibers (e.g. woodpulp fluff), synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the absorbent body may include one or more components that can modify menses or intermenstrual liquid.

In a particular arrangement, the intake layer can be a thermally-bonded, stabilized airlaid fibrous web (e.g. Concert code 175.1020) available from Concert Fabrication, a business having offices located in Gatineaux, Quebec, Canada. The intake layer may optionally be provided by a similar, stabilized airlaid fibrous web available from Buckeye Technologies, Inc., a business having offices located in Memphis, Tenn. U.S.A.

In a desired feature, the intake layer 32 can have a relatively lower basis weight, as compared to the bottom (garment-side) retention/shaping layer 36. Optionally, the basis weight of the intake layer may be equal or similar to the basis weight of the shaping layer. In another feature, the intake layer 32 can have a lower density (e.g., be more lofty), as compared to the retention/shaping layer 36. Alternatively, the basis weight of the intake layer can be higher than or equal to the basis weight of the shaping/retention layer 36.

In a particular aspect, the basis weight of the intake layer 32 can be at least a minimum of about 30 g/m². The basis weight of the intake layer can alternatively be at least about 100 g/m², and can optionally be at least about 120 g/m² to provide improved performance. In other aspects, the basis weight of the intake layer can be up to a maximum of about 250 g/m², or more. The basis weight of the intake layer can alternatively be up to about 200 g/m², and can optionally be up to about 175 g/m² to provide improved effectiveness.

In a desired feature, the top (bodyside) intake layer 32 of the present invention can be smaller in size than the bottom retention/shaping layer 36. Accordingly, the bottom retention/shaping layer 36 can be larger than the top intake layer, and can substantially define the overall size of the absorbent body 30. Optionally, the bottom retention/shaping layer 36 can be substantially equal to, or relatively smaller than the top intake layer.

The intake layer can be substantially centered (in its machine-direction and cross-direction) with respect to the shaping layer. Optionally, the intake layer may be skewed or offset in one direction (e.g. along the machine-direction), depending on where liquid is expected to first enter the absorbent article.

The top intake layer 32 may have any operative shape and/or design. For example, the intake layer may include a single piece of material, or multiple pieces of material. For example, the intake layer may include multiple strips of material. In addition, the intake layer 32 may include holes or apertures to better provide desired liquid-intake properties. The apertures may extend partially or completely through the z-directional thickness of the intake layer 32, as desired.

The shaping layer 36 can provide the functions of liquid storage and retention, liquid distribution, liquid spreading and shape maintenance. The shaping layer may include natural fibers (e.g. woodpulp fluff), synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the shaping layer may include one or more components that can modify the menses or intermenstrual liquid.

In a particular arrangement, the shaping layer can be a thermally-bonded, stabilized airlaid fibrous web available from Concert Fabrication (Concert code 225.1021), a business having offices located in Gatineaux, Quebec, Canada (e.g. Concert code 225.1021). The shaping layer 36 may optionally be provided by a similar, stabilized airlaid fibrous web available from Buckeye Technologies, Inc., a business having offices located in Memphis, Tenn., U.S.A.

In a particular aspect, the basis weight of the shaping layer 36 can be at least a minimum of about 30 g/m² or 100 g/m². The shaping layer basis weight can alternatively be at least about 130 g/m², and can optionally be at least about 165 g/m² to provide improved performance. In other aspects, the basis weight of the shaping layer can be up to a maximum of about 400 g/m², or more. The shaping layer basis weight can alternatively be up to about 350 g/m², and can optionally be up to about 325 g/m² to provide improved effectiveness. In a desired configuration, the shaping layer basis weight can be about 225 g/m².

Further details regarding a suitable absorbent and absorbent system are described in U.S. Patent Application Publication 2004/0186448, which was published Sep. 23, 2004 (attorney docket No. 18997). The entire disclosure of this document is incorporated herein by reference in a manner that is consistent herewith.

As representatively shown, the personal care article 20 can further include a garment-attachment mechanism operatively connected to a garment-side of the backsheet layer 28. In a desired feature, a selected configuration of a garment-attachment mechanism may be distributed onto the garment-side of the article to help operatively secure the article to a wearer's outer-garment. Any operative fastening or attachment mechanism may be employed. The garment-attachment mechanism may, for example, include a hook-and-loop fastener, an interengaging mechanical fastener, a cohesive fastener, an adhesive fastener or the like, as well as combinations thereof. In particular aspects the garment-attachment mechanism can include an adhesive 38. The adhesive may be pressure-sensitive, and may have a configuration that adheres well to textile materials, but adheres poorly to a wearer's skin. The garment-attachment mechanism can be arranged in any operative configuration, such as the illustrated strip regions. Typically, the garment adhesive can be distributed over the garment-side of the backsheet, and one or more layers or sheets of release material 40 can be removably placed over the garment adhesive during storage prior to use.

In desired arrangements, the amount of garment adhesive can be relatively small. For example, the garment adhesive 38 can have the configuration of a continuous central strip, which extends along approximately the entire length of the article and has cross-directional width of about 5 mm.

In optional arrangements, the article 20 may include additional components or component layers, as desired. For example, a transfer layer may be positioned between the intake layer 32 and the shaping layer 36. In another feature, the article may include any desired pattern of embossments formed into at least the bodyside surface of the article. The embossing can deform the bodyside of the topsheet and can deform selected portions of the absorbent body 30 to provide operative channel regions that can help block, direct or otherwise control a desired movement of liquids along the bodyside surface of the article. The embossing can also provide an aesthetic benefit to the consumer, and a visual cue regarding fit and leakage protection. In particular arrangements, the embossments can be positioned generally adjacent the perimeter edges of the absorbent body 30. In other aspects, the embossments can be configured to provide a regular or irregular pattern having one or more channels which are distributed in a symmetrical or asymmetrical array, as desired.

In another feature, any of the components of the article 20 may include a fragrance or scented material. For example, a fragrance or scented material may be incorporated in the topsheet 26 and/or backsheet 28 and/or absorbent body 30. In a desired arrangement, the scented material can be included in the backsheet to provide a more simplified manufacturing process, and to provide a configuration that more effectively separates the scent from the wearer's skin.

The fragrance may be encapsulated or non-encapsulated, and any operative fragrance or scented material may be employed. Examples of suitable fragrances can include single floral-type fragrances such as lavender, lilac, rose and jasmine; floral bouquet-type fragrances, which are a mixture of single floral-type fragrances; citrus-type fragrances such as lemon or orange; oriental-type fragrances such as musk, amber and civet; green or woody-type fragrances; aliphatic aldehyde-type fragrances; and the like; as well as combinations thereof. In desired arrangements, single floral-type fragrances such as lavender, lilac and rose can be employed.

For example, a suitable lavender fragrance can have the following composition: lavender oil 40 to 60%, linalool or linalool oxide 10 to 30%, citronelol 5 to 10%, isobornyl acetate 10 to 20% and camphor 5 to 10%. The camphor can help to slightly intensifies the lavender scent. Another lavender fragrance can have the following composition: linalyl acetate 25 to 40%, lavandin oil 40 to 60%, linalool or linalool oxide 5 to 15% and lemon oil 0.01 to 5%.

A suitable lilac fragrance can have the following composition: cinnamic aldehyde 30 to 60%, ethyl linalool 10 to 30%, iso-eugenol 10 to 30%, linalool 10 to 30%, terpineol 10 to 300% and phenyl ethyl alcohol 5 to 15%.

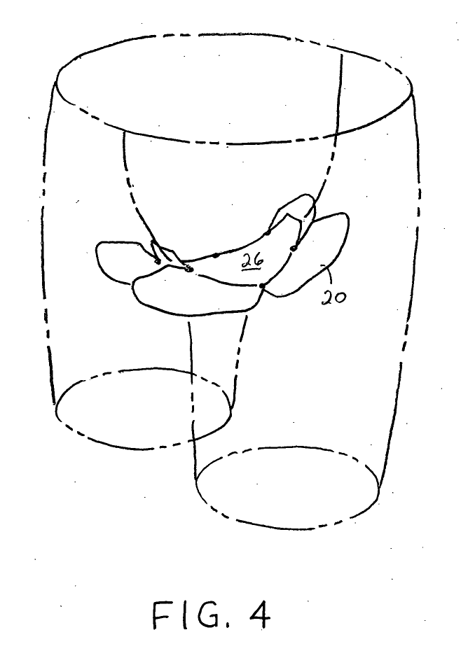

There has been a growing trend where young independent users of personal care pad articles are occasionally “going commando”. The users not wearing underwear when wearing close fitting clothing to avoid the appearance of the outlines of the underwear. Conventional pantiliners or pads, however, fit poorly into the crotch region of ordinary pants, thigh pants or sport pants due to the way those pants-type outer-garments are cut and sewn. Thus, a sufficiently effective, direct protection of a user's outer clothing from staining or soiling remains an unmet need. With reference to FIG. 4, the configurations of the article 20 can help provide a distinctive personal care, garment article that can more effectively be attached directly to the crotch area in a pair of pants without the use of an intervening undergarment. The pants-type of outer garment can be any type of close-fitting clothing worn against the crotch area of the outer garment. Optionally, the article 20 may be employed in combination with a conventional undergarment, or in combination with conventional sanitary protection products, such as sanitary pads and tampons, to further protect the clothing from staining.

Typically, the crotch area of the outer garment does not include a generally flat area that would accommodate an ordinary type of protective liner. To the contrary, pants-types of clothing, such as trousers, shorty pants, sport pants, tights, sweat pants, pantyhose and the like, are typically constructed with two major seams. A crotch seam extends between the legs of the pants and runs from the rear waistband to the front waistband of the pants. A leg inseam extends from the distal end of one pants leg, through the crotch region of the pants, and to the distal end of the second pants leg.

The article 20 can be configured with a distinctive geometry and stay-in-place system to more effectively accommodate the concave and convex lines and surfaces in the pants garment that arise from the presence of the crotch seam and leg inseam. As a result, the article 20 can achieve a better fit in the various types of pants, can more easily be placed and positioned within the pants, and can more effectively maintain a desired positioning and location within the pants. Accordingly, the article can better provide desired stay-in-place performance and wearing comfort. In particular arrangements, the article 20 may, for example, include a system of slits and notches to help the article follow the curvature of the seams of the pants, and help improve the engagement between the article and the pants seams.

The representatively shown system of notches and lines of bending weakness and flexibility (e.g. fold lines) can help facilitate a desired placement of the article 20 in the crotch area of the user's pants. The notches can operatively engage the inner crotch seams and inner leg seams of the crotch region of the wearer's pants. The notches can also help provide a visual cue for proper placement of the article 20 in the pants of the wearer. The flexible lines of bending weakness can help the article conform to the inner shapes of the wearer's pants.

With reference to FIGS. 1, 2 and 4, the first end-notch 80 can have a first end-notch length 84 which extends inward from the perimeter 34 and into the area encompassed by the article 20. The first end-notch 80 can also have a width 86 which extends perpendicular to the notch length. Additionally, the second end-notch 82 can have a second end-notch length and a second end-notch width which are equal to or different than the length and width of the first end-notch 80. For example, the notch-width 86 can be relatively shorter or relatively longer than the corresponding notch-length 84. The end-notches are configured to operatively engage the anterior and posterior regions of the inner crotch seam of the wearer's selected outer-garment. Accordingly, the end-notches can help align the article 20 along the centerline of the wearer's body, and can help reduce undesired, side-to-side movements of the article 20 relative to the wearer's outer-garment.

In particular aspects, the length 84 of an individual end-notch can be at least a minimum of about 10 mm. The end-notch length can alternatively be at least about 15 mm, and can optionally be at least about 25 mm to provide desired benefits. In other aspects, the end-notch length can be up to a maximum of about 50 mm, or more. The end-notch length can alternatively be up to about 40 mm, and can optionally be up to about 35 mm to provide desired effectiveness. In a desired arrangement, the end-notch length can be about 28 mm.

In other particular aspects, the width 86 of an individual end-notch can be at least a minimum of about 10 mm. The end-notch width can alternatively be at least about 20 mm, and can optionally be at least about 25 mm to provide desired benefits. In other aspects, the end-notch width can be up to a maximum of about 50 mm, or more. The end-notch width can alternatively be up to about 40 mm, and can optionally be up to about 35 mm to provide desired effectiveness. In a desired arrangement, the end-notch width can be about 32 mm.

Similarly, side-notches can be configured to operatively engage the left and right, inner leg seams of the wearer's selected outer-garment. Accordingly, the side-notches can help align the article 20 along the side-to-side direction of the wearer's body, and can help reduce undesired, fore-and-aft movements of the article 20 relative to the wearer's outer-garment.

As representatively shown, the first side-notch 66 and the second side-notch 70 can each have a corresponding individual notch length 84, which extends inward from the perimeter 34 of the article 20, and a corresponding notch width 86. It should be readily appreciated that the length and/or width of the first side-notch 66 can be different than or substantially equal to the length and/or width of the second side-notch 70. In particular aspects, the length 84 of an individual side-notch can be at least a minimum of about 0.1 mm. The side-notch length can alternatively be at least about 5 mm, and can optionally be at least about 10 mm to provide desired benefits. In other aspects, the side-notch length can be up to a maximum of about 25 mm, or more. The side-notch length can alternatively be up to about 20 mm, and can optionally be up to about 15 mm to provide desired effectiveness. In a desired arrangement, the side-notch length can be about 12 mm:

In a further aspect, the width 86 of the individual side-notch can be at least a minimum of about 0.1 mm. The side-notch width can alternatively be at least about 5 mm, and can optionally be at least about 10 mm to provide desired benefits. In other aspects, the side-notch width can be up to a maximum of about 25 mm, or more. The side-notch width can alternatively be up to about 20 mm, and can optionally be up to about 15 mm to provide desired effectiveness. In a desired arrangement, the side-notch width can be about 12 mm.

An individual notch (80, 82, 66, 70) can be configured to project and extend into the topsheet layer 26 and the backsheet layer 28, and through the thickness dimensions of the topsheet and backsheet layers. Additionally, the individual notch can be configured to project and extend into the absorbent body 30 that is incorporated into the article 20. An individual notch can be tapered or non-tapered, as desired. As representatively shown, the notch can have a generally triangular or a generally trapezoidal shape.

At least a first, end-line of bending weakness 88 can be configured to extend from an inboard, apex end region of the first end-notch 80 and into the interior of the area of the article. As representatively shown, for example, the first, end-line of bending weakness can include a first, end-slit 88 which extends from the inboard, apex end region of the first end-notch 80. The personal care article 20 can further include a second end-line of bending weakness which extends from an inboard, apex end region of the second end-notch 82. As representatively shown, the second end-line of bending weakness can include a second end-slit 92 which extends from the inboard, apex end of the second end-notch 82.

The personal care article 20 can further include a first, complementary, end-line of bending weakness which extends from the inboard, apex end region of the first end-notch 80. The first complementary, end-line of bending weakness can, for example, include a first, complementary end-slit 90 which extends from the inboard, apex end region of the first end-notch. Additionally, the article 20 can include a second, complementary, end-line of bending weakness which extends from the inboard, apex end region of the second end notch 82. In a particular configuration, the second, complementary, end-line of bending weakness can include a second, complementary, end-slit 92 which extends from the inboard, apex end region of the second end-notch 82.

As representatively shown, an individual end-line of bending weakness can have a width and a corresponding length, with the width of the end-line being relatively shorter than the corresponding length of the end-line. For example, an individual end-slit can have a slit-width 100 and a slit-length 96, with the slit-width being relatively shorter than the slit-length. In particular aspects, the end-line width (e.g. slit-width 100) can be a minimum of about 0.1 mm, or less. The end-line width can alternatively be at least about 0.2 mm, and can optionally be at least about 0.3 mm to provide desired benefits. In other aspects, the end-line width can be up to a maximum of about 5 mm, or more. The end-line width can alternatively be up to about 2 mm, and can optionally be up to about 1 mm to provide desired effectiveness.

In another aspect, the end-line length (e.g. length 96 of an end-slit) can be at least a minimum of about 1 mm. The end-line length can alternatively be at least about 3 mm, and can optionally be at least about 5 mm to provide desired benefits. In other aspects, the end-line length can be up to a maximum of about 30 mm, or more. The end-line length can alternatively be up to about 25 mm, and can optionally be up to about 20 mm to provide desired effectiveness.

An individual end-slit or other end-line of bending weakness can have a selected end-line (e.g. slit) angle 98. As representatively shown, the end-line angle is an acute angle which is measured or-determined from a line that is parallel to the longitudinal centerline 52 of the article 20. In particular aspects, the end-line angle can be at least a minimum of about 20 degrees. The end-line angle can alternatively be at least about 25 degrees, and can optionally be at least about 30 degrees to provide desired benefits. In other aspects, the end-line angle can be up to a maximum of about 45 degrees, or more. The end-line angle can alternatively be up to about 40 degrees, and can optionally be up to about 35 degrees to provide desired effectiveness. If the angle 98 is outside the desired values, the article can be less able to provide desired levels of conformance with the wearer's pants garment

The personal care article 20 may optionally include a first side-slit 54 or other side-line of bending weakness, which extends laterally inboard from the inboard, apex end region of the first side-notch 66. Similarly, the article can optionally include a second side-slit 56 or other side-line of bending weakness, which extends laterally inboard from the inboard, apex end region of the second side-notch 70. As representatively shown, an individual side-line of bending weakness can have a width and a corresponding length, with the width of the side-line width being relatively shorter than the length of the side-line. For example, an individual side-slit can have a slit-width 100 and a slit-length 96, with the slit-width being relatively shorter than the slit-length. In particular aspects, the side-line width (e.g. slit-width 100) can be a minimum of about 0.1 mm, or less. The side-line width can alternatively be at least about 0.2 mm, and can optionally be at least about 0.3 mm to provide desired benefits. In other aspects, the side-line width can be up to a maximum of about 5 mm, or more. The side-line width can alternatively be up to about 2 mm, and can optionally be up to about 1 mm to provide desired effectiveness.

In another aspect, the side-line length (e.g. length 96 of a side-slit) can be at least a minimum of about 1 mm. The side-line length can alternatively be at least about 3 mm, and can optionally be at least about 5 mm to provide desired benefits. In other aspects, the side-line length can be up to a maximum of about 30 mm, or more. The side-line length can alternatively be up to about 25 mm, and can optionally be up to about 20 mm to provide desired effectiveness.

An individual line of bending weakness can be configured to extend into a limited portion of the longitudinal and lateral dimensions of the article 20. Additionally, the line of bending weakness can be configured to extend partially or entirely through the thickness dimension of the article. In a particular aspect, the line of bending weakness can be configured to provide a line of increased flexibility which facilitates a pivoting or hinging action about the line of bending weakness. Accordingly, the lines of bending weakness can help the article twist and bend to conform to the interior shapes and dimensions of the wearer's outer-garment. In another aspect, the line of bending weakness can be configured to an operative amount of expandability or extensibility, at least along a direction that is generally perpendicular to the length dimension of the individual line of bending weakness. The extensibility may, for example, be provided by a void region, an embossed region, a region that includes a region of material that is operatively extensible or the like, as well as combinations thereof.

An individual end-slit, side-slit or other line of bending weakness can be substantially linear or straight. Alternatively, each end-slit, side-slit or other line of bending weakness can be linear, non-linear, curvilinear or the like, as well as combinations thereof.

In a further feature, the article 20 can include a distinctive system of tear-stops. As representatively shown, a corresponding tear-stop can be located at an inboard, terminal end region of each corresponding line of bending weakness. As representatively shown, for example, an end-slit tear-stop 102 can be located at an inboard, terminal end region of each corresponding end-slit. Accordingly, a first, end-slit tear-stop 102 can be located at an inboard, terminal end region of the first end-slit 88, and a second, end-slit tear-stop 102 can be located at an inboard, terminal end region of the second end-slit 92. Similarly, a first, complementary, end-slit tear-stop can be located at an inboard, terminal end region of the first, complementary end-slit 90, and a second, complementary, end-slit tear-stop can be located at an inboard, terminal end region of the second, complementary end-slit 94. In a like manner, a first side-slit tear-stop 102a can be located at an inboard, terminal end region of the first side-slit 54, and a second side-slit tear-stop 102a can be located at an inboard, terminal end region of the second side-slit 56.

An individual tear-stop 102 can be provided by any operative mechanism. The tear-stop can, for example, include or be provided by an area of meltblown adhesive, an area of thermal embossing, a cut-out or other void region with a contoured terminal edge having a relatively enlarged radius of curvature that operatively reduces stress concentrations at the inboard end of the corresponding slit, or the like, as well as combinations thereof. An individual tear-stop can have any operative shape. The shape can, for example, be substantially circular, oval, rectilinear or the like, as well as combinations thereof.

Additionally, an individual tear-stop can have a cross-dimension (e.g. cross-slit dimension) which is greater than the width of its corresponding line of bending weakness (e.g. slit line). The cross-dimension of an individual tear-stop 102 is measured along a dimension that is substantially perpendicular to the length dimension of the corresponding line of bending weakness. In particular aspects, the width 104 of the tear-stop can be at least a minimum of about 0.1 mm. The tear-stop width can alternatively be at least about 0.2 mm, and can optionally be at least about 0.3 mm to provide desired benefits. In other aspects, the tear-stop width can be up to a maximum of about 5 mm, or more. The tear-stop width can alternatively be up to about 2 mm, and can optionally be up to about 1 mm to provide desired effectiveness.

An individual tear-stop 102 can provide an enhanced tear-strength value at the location of the region of the article that includes the tear-stop. The enhanced tear-strength can desirably be greater than or at least equal to a tear strength value of the article at a substantially equivalent region of the article that includes an end-slit or side-slit, but does not include a corresponding tear-stop structure. In particular aspects, the tear-stop can provide a tear-strength value which is increased by an amount which is at least a minimum of about 1 Newtons (N), as compared to a substantially equivalent region of the article that includes an end-slit or side-slit, but does not include a corresponding tear-stop structure. The tear-stop can alternatively provide an enhanced tear-strength value which is increased by at least about 1.5 N, and can optionally provide an enhanced tear-strength value which is increased by at least about 2 N to provide desired benefits. In other aspects, the tear-stop can provide an enhanced tear-strength value which is increased by 75 N, or more. The tear-stop can alternatively provide an enhanced tear-strength value which is increased by up to about 60 N, and can optionally provide an enhanced tear-strength value which is increased by up to about 50 N to provide desired effectiveness.

In still other aspects, the individual tear-stop can provide a tear-strength value which is increased by at least a minimum of about 10%, as compared to a substantially equivalent region of the article that includes an end-slit or side-slit, but does not include a corresponding tear-stop structure. The tear-stop can alternatively provide an enhanced tear-strength value which is increased by at least about 13%, and can optionally provide an enhanced tear-strength value which is increased by at least about 15% to provide desired benefits. In further aspects, the tear-stop can provide an enhanced tear-strength value which is increased by 75%, or more. The tear-stop can alternatively provide an enhanced tear-strength value which is increased by up to about 60%, and can optionally provide an enhanced tear-strength value which is increased by up to about 50% to provide desired effectiveness.

A suitable technique for determining the tear strength value of the tear-stop is described in the Examples section of the present disclosure. The Examples section also provides representative determinations of the enhanced tear strength that can be provided, by the incorporation of various configurations of tear-stops.

In another feature, the article 20 can include a distinctively configured stay-in-place system. The stay-in-place system may, for example, include a generally central strip of garment adhesive 38 which operatively secures the article 20 in the user's pants. Additionally, the stay-in-place system can include a distinctive anti-slip material that is located on and operatively secured to a major portion of the appointed garment-side of the backsheet layer 28. As a result, the article can provide a better, close-fit to the user's pants, and can better maintain a desired positioning of the article 20 in the crotch and upper leg areas of the pants. The slip-resistant backsheet materials can have a significantly high coefficient of friction with respect to the clothing materials of the user's pants, and the high coefficient of friction can help provide an increased engagement that can enhance the operation of the stay-in-place mechanism. The garment adhesive strip can help to initially provide the desired placement of the article 20 in the crotch region of the user's pants. The combination with the slip-resistant material can help reduce the amount of the employed garment adhesive 38.

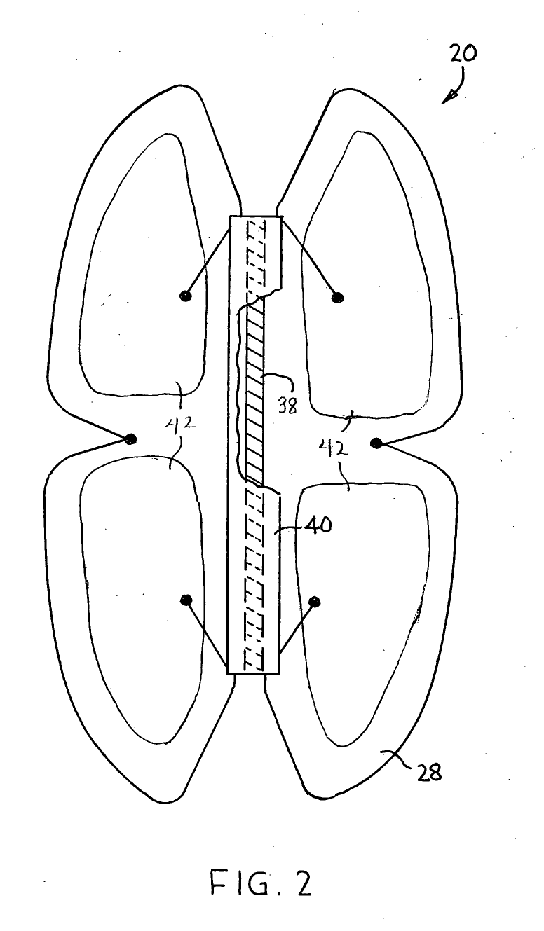

With reference to FIG. 2, the article 20 can include a slip-resistant, garment-engagement mechanism 42 which is attached, combined or otherwise operatively connected to a garment-side of the backsheet layer 28. The garment-engagement mechanism may be distributed over an entire garment-facing surface of the backsheet, or may be distributed over a selected portion of the garment-facing surface of the backsheet in an operative array or pattern. In particular arrangements, the employed slip-resistant material may be configured to have a tacky or rubbery feel. Examples of suitable slip-resistant materials can include: a layer of meltblown elastomeric fibers, other rubbery materials, a low-tack adhesive, materials with highly-textured skid-resistant surfaces, materials having a multiplicity of miniature spike-type protrusions, a dry, strong, reversible, self-cleaning adhesive that has been referred to as “gecko feet” materials, or the like, as well as combinations thereof. Particular examples of slip-resistant materials that can be attached to or otherwise operatively combined with the backsheet 28 can include KRATON G polymers type SEBS, (e.g. KRATON type G1657) with 0 to 20% tackifier. Other slip-resistant materials could include a KRATON 1107 material, or a SOLPRENE 420 material. Other examples may include an adhesive having a low peel strength, such as the adhesive employed on POST-IT notes. For example, a thin meltblown layer which has a basis weight within the range of about 30-60 gsm and includes elastomeric fibers can help provide anti-slip properties. Operative coatings of other anti-slip materials can also be applied using meltblown technology to provide a high level of slip resistance when contacted against textile materials.

In a particular feature, the garment-engagement mechanism 42 can be configured to provide a high coefficient-of-friction value to provide a more effective engagement with the material of the wearer's outer garment. In particular aspects, the static coefficient-of-friction value can be at least a minimum of about 0.5. The static coefficient-of-friction value can alternatively be at least about 0.7, and can optionally be at least about 1 to provide desired benefits. In other aspects, the static coefficient-of-friction value can be up to a maximum of about 10, or more. The static coefficient-of-friction value can alternatively be up to about 7, and can optionally be up to about 4 to provide desired performance.

In further aspects, the kinetic or dynamic coefficient-of-friction value of the garment-engagement mechanism can be at least a minimum of about 0.5. The dynamic coefficient-of-friction value can alternatively be at least about 0.7, and can optionally be at least about 1 to provide desired benefits. In other aspects, the dynamic coefficient-of-friction value can be up to a maximum of about 9, or more. The dynamic coefficient-of-friction value can alternatively be up to about 6, and can optionally be up to about 3 to provide desired effectiveness.

If the coefficient-of-friction values are outside the desired values, the garment-engagement mechanism may not provide adequate levels of slip-resistance, and may move excessively during use. For determining the coefficient-of-friction values of the garment-engagement mechanism, a suitable technique is described in the Examples section of the present disclosure.

In another feature, the garment-engagement mechanism can be configured to provide a low-peel-strength value to help provide the desired engagement with a wearer's outer garment (e.g. a pants-type outer garment). In a particular aspect, the peel-strength value provided by the garment-engagement mechanism can be at least a minimum of about 0.1 g. The peel-strength value can alternatively be at least about 0.5 g, and can optionally be at least about 0.7 g to provide desired benefits. In other aspects, the peel-strength value of the garment-engagement mechanism can be up to a maximum of about 30 g. The peel-strength value can alternatively be up to about 25 g, and can optionally be up to about 20 g to provide desired effectiveness. If the peel-strength value is outside the desired values, the garment-engagement mechanism may excessively change position or may excessively adhere to the wearer's outer garment. A suitable technique for determining the peel-strength of the garment-engagement mechanism is described in the Examples section of the present disclosure.

Since discretion can be an important feature, the personal care article can be configured with distinctive length, width and thickness dimensions. To help allow the article 20 to be discreetly positioned in the wearer's outer garment, the article 20 can have an article length 46 which is at least a minimum of about 80 mm. The article length can alternatively be at least about 110 mm, and can optionally be at least about 140 mm to provide desired benefits. In other aspects, the article length can be up to a maximum of about 200 mm, or more. The article length can alternatively be up to about 180 mm, and can optionally be up to about 160 mm to provide desired discretion and effectiveness.

Additionally, the article 20 can have an article width 48 which is at least a minimum of about 60 mm. The article width can alternatively be at least about 70 mm, and can optionally be at least about 80 mm to provide desired benefits. In other aspects, the article width can be up to a maximum of about 120 mm, or more. The article width can alternatively be up to about 100 mm, and can optionally be up to about 90 mm to provide desired performance.

Additionally, the article 20 can have an article thickness 50 which is at least a minimum of about 0.2 mm. The article thickness can alternatively be at least about 0.3 mm, and can optionally be at least about 0.5 mm to provide [improved] desired benefits. In other aspects, the article thickness can be up to a maximum of about 1.5 mm, or more. The article thickness can alternatively be up to about 1.2 mm, and can optionally be up to about 1 mm to help the article 20 remain discreet and unobtrusive.

To help the performance of the personal care article 20, the article can be constructed with materials that are highly drapeable. Additionally, the materials can be configured to extensible one or two directions. For example, the materials can be configured to be elastomerically stretchable along one or more directions. The article may also be configured to be breathable or otherwise vapor-permeable. In optional configurations, the article can be configured to be flushable in an ordinary toilet. Selected portions of the article 20 may be configured to be transparent or translucent. Alternatively, the article can be configured to have a discreet color. To accommodate a range of clothing sizes, the article can be produced in several alternative sizes. While the article will typically be used intermenstrually, it should be appreciated that the article may, on occasion, be used in combination with other feminine care articles, such as tampons.

The highly flexible and drapeable configurations of the article 20, can help provide a closer fit with the shape and geometry of the user's outer pants garment, and can help provide increased comfort. The article 20 can, for example, help avoid discomfort linked to the folding or creasing of the article, the undesired self-sticking between different parts of the article, or the undesired sticking of the article to the upper legs or other parts of the user's body.

### EXAMPLES

Tear Strength—Trapezoid Tear

Individual specimens of five types of samples were prepared for tear strength evaluation. Each sample-type had a particular configuration of the tear-stop structure. Each specimen was a laminate which contained a 85 g/m² layer of CONCERT MH085.103, stabilized airlaid fibrous material; 3 g/m²of FULLER HL 1455 construction adhesive; and a 20 micrometer (μm) thick layer of HUHTAMAKI 30901 polymer film. Each specimen was pre-slit with a 20 mm long slit. The configuration of the tear-stop structure in each sample-type is summarized below:
Code N: Null sample, slit only, no tear-stop.
Code C: Circular disk of material has been separated and removed from the sample at the innermost end of the slit to provide a tear-stop structure.
Code H: Half-Circle shaped piece of material has been separated and removed from the sample at the innermost end of the slit to provide a tear-stop structure.
Code A: a region of INSTANT PAK 2600 adhesive wax has been applied at the innermost end of the slit to provide a tear-stop. The INSTANT PAK adhesive wax is available from National Starch and Chemical Company, a business having offices located in Bridgewater, N.J., U.S.A.
Code T: a region of thermo-embossing (120 C.) has been formed at the innermost end of the slit to provide a tear-stop.

The specimens were tested with the following Trapezoid Tear Procedure to determine the tear strength values provided by their corresponding tear-stop structures.

Tear Strength (Trapezoid Tear) Procedure

Employing ASTM D 5733-99, individual specimens of five tear-stop sample codes were evaluated for their tear strength values relative to a control that did not include a tear-stop structure. The specimens were cut to approximately 3×6 inches (approximately 7.6×15.2 cm), and were pre-slit with a slit length of 20 mm instead of the 15 mm length specified in ASTM D 5733-99, and were referenced with a marking template to the dimensions specified in the ASTM method.

Specimens were conditioned for 24 hours at 23° C. and 50% relative humidity (RH) prior to testing in the same environment. A SINTECH model 1/G constant rate of extension, tensile frame (available from MTS Systems Corporation, a business having offices located in Eden Prairie, Minn., U.S.A.), was used for the testing. Alternatively, a substantially equivalent system may be employed. The gage length was 1 inch (2.54 cm), and the frame speed was 300 mm per minute. The test end point was 50 mm of crosshead travel, which equated to approximately 10 mm of tear-length beyond the end of the slit.

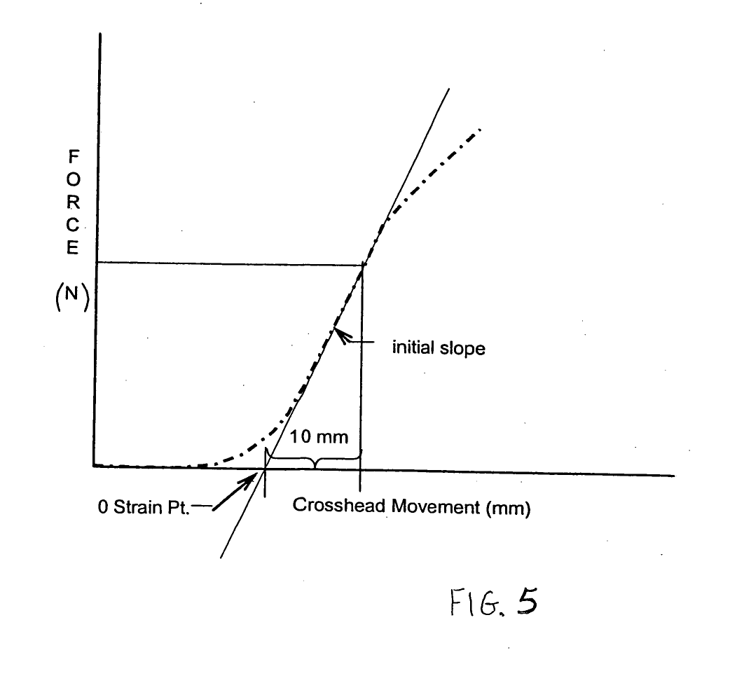

The test data from an individual specimen is plotted on a graph which has the cross-head movement (millimeters) on the horizontal x-axis, and has the force (Newtons) on the vertical y-axis. A representative graph is illustrated in FIG. 5. Due to the stretching deformation of the specimens, and the different sizes of the tear-stops, a common stopping-point was not employed to determine the strength values of the tear-stops. To determine the strength value of a tear-stop specimen, a line is drawn along the slope initial tensile force data and back to a point of intersection with the x-axis (zero strain point), as illustrated in FIG. 5. Then a crosshead movement point that that corresponds to 10 mm of crosshead travel beyond the zero strain point is identified on the graph. On the plotted data, the force value that corresponds to the 10 mm of crosshead travel beyond the zero strain point is then identified as the tear strength of the tear-stop specimen. The identified force value data from 5 specimens is arithmetically averaged to determine a tear strength value of the sample. Data from the Trapezoid Tear testing are summarized in the following Table 1.

TABLE 1
Trapezoid Tear (Newtons), n = 5

| | | Average | | | | |
|---|---|---|---|---|---|---|
| Code | Tear-Stop Type | (N) | s | COV (%) | Δ1 (N) | Δ2 (%) |
| N | Control | 8.8 | 0.9 | 9.7 | — | — |
| A | Adhesive Wax | 13.1 | 2.3 | 17.8 | 4.3 | 49 |
| H | Half-Circle, | 11.6 | 2.9 | 25.0 | 2.8 | 32 |
| | void-area | | | | | |
| C | Circle, void-area | 10.3 | 0.5 | 4.5 | 1.5 | 17 |
| T | Thermo-Embossment | 10.3 | 2.1 | 20.0 | 1.5 | 17 |

In the Tables:
s = standard deviation.
COV = coefficient of variance.
Δ1 = increase in tear strength (Newtons), compared to control code N;
where: Δ1 = tear strength value (Newtons) of code with tear-stop, minus the tear strength value of control code N with no tear-stop.
Δ2 = % increase in tear strength, compared to control code N;
where: Δ2 = 100% * Δ1(Newtons) ÷ (tear strength value of control code N).

Coefficient-of-Friction and Peel Strength (T-Peel)

Individual specimens of four types of coated-film samples were prepared for coefficient-of-friction (COF) testing and peel adhesion (T-Peel) testing against a layer of standard cotton fabric. The standard cotton fabric was a black, knit cotton material which is a 100% cotton jersey material, black, with no surface treatment, weight is 6.8 OSY (about 230 g/m²). Rolls of the fabric can be purchased at a web width of about 60-62 inch (about 152-157 cm), with a weight per roll of approximately 60 lbs (about 27 Kg). The standard knit cotton fabric material is available from Contempora Fabrics, Inc., a business having offices located in Lumberton, N.C., U.S.A., and is identified with the part number, STYLE 808 BLACK 6175.

In each specimen, a layer of HYFOL polypropylene (PP) film was coated with a slip-resistant coating material applied onto one side of the film, and the width of the applied coating was about 90-93 mm. The coating material was configured to provide a garment-engagement mechanism.

The configuration of each sample-type is summarized below:
PP film only: HYFOL polypropylene (PP) film, 20/μm thickness, no coating.
Q5432: HYFOL PP film with TECHNOMELT Q-5432 coating, with a coating basis weight of 25 g/m².
PS 578: HYFOL PP film with TECHNOMELT PS 578 S coating, with a coating basis weight of 25 g/m².
PS 500: HYFOL PP film with TECHNOMELT SUPRA PS 500 coating, with a coating basis weight of 25 g/m².

The HYFOL PP film is available from RKW AG, Film Division, a business having offices located in Liege, Belgium. The TECHNOMELT coating materials are available from Henkel Technologies, a business having offices located in Dusseldorf, Germany.

The coated specimens were tested with the following COF Procedure to determine the coefficient-of-friction values provided by their corresponding coating materials.

COF Test Procedure

Static and kinetic coefficient-of-friction (COF) values of the coated specimens were determined against a knit cotton standard at 150 mm/minute on a LAB MASTER Slip & Friction Tester, Model 32-90, which was obtained from Testing Machines, Inc., a business having offices located in Ronkonkoma, N.Y., U.S.A. Alternatively, a substantially equivalent system may be employed. The COF testing was conducted in accordance with ASTM 1894-95, “Standard Method for Static and Kinetic Coefficients of Friction of Plastic Film and Sheeting”. Four samples codes, three coated embossed film samples (coated on the female side) and one uncoated film control were cut to the dimensions of 3.5 inches (8.9 cm) wide×4 inches (10.2 cm) long. The standard cotton material was cut to the dimensions of 5 inches (12.7 cm) wide×10 inches (25.4 cm) long. Samples were then conditioned for 24 hours at 23° C. and 50% relative humidity prior to testing in the same environment.

Two, 9 inch (22.9 cm) long strips of a two-sided tape 0.005 inches (0.013 cm) thick were applied to the tester bed, side-by-side, with one tape butted to the other with no overlap. The standard cotton fabric was placed onto the tape with the wale side up, and the wale oriented parallel to the length of the bed. A standard 4.5 lb (2.04 Kg) PSTC (Pressure Sensitive Tape Council) hand roller (with no added hand pressure) was used to attach the cotton fabric to the tape. Details regarding the hand roller can be found in Appendix B of the 14th Edition of the Pressure Sensitive Tape Counsel Methods). The attachment between the cotton fabric and the tape was achieved by rolling across the wale in a slightly overlapping zig-zag fashion, from one end of the cotton fabric to the other, then back again. Specimens of the coated film were attached to the bottom of the sled with the coated side of the film facing down, away from the sled. In the case of the coated specimens, the coating extended the full width of the 2.5 inch (1.3 cm) wide sled. Three specimens per sample code were tested, and the arithmetic average of the COF data from three specimens provided a COF value for the sample. Data from the coefficient-of-friction testing are summarized below in Table 2 and Table 3.

TABLE 2
COF (Static), n = 3

| Code | Average | s | COV (%) |
|---|---|---|---|
| PP film only | 0.46 | 0.05 | 9.87 |
| PS-500 | 1.72 | 0.08 | 4.87 |
| PS-578 | 3.11 | 0.21 | 6.63 |
| Q-5432 | 2.00 | 0.10 | 5.00 |

TABLE 3
COF (Kinetic), n = 3

| Code | Average | s | COV (%) |
|---|---|---|---|
| PP film only | 0.45 | 0.40 | 9.05 |
| PS-500 | 1.78 | 0.09 | 4.99 |
| PS-578 | 2.90 | 0.07 | 2.30 |
| Q-5432 | 2.14 | 0.06 | 3.00 |

T-Peel Procedure

The T-Peel testing was conducted in accordance with ASTM D1876 01, “Standard Test Method for Peel Resistance of Adhesives”, with modifications. A SINTECH Model 1/G constant rate of extension tensile frame (available from MTS Systems Corp.), was used for the testing. Alternatively, a substantially equivalent system may be employed. The tensile frame was set up with a 10 N load cell, and light 1×3 inch (2.54×7.6 cm) rubber-faced grips, such as INSTRON grips, 75 psi (517 KPa) maximum, were installed after load cell calibration. The gage length was set at 2 inch (5.1 cm), and the crosshead speed set to 127 mm/minute. The software program employed with the testing system was configured to begin collecting peel data after 13 mm of peel (26 mm of cross-head travel) and to stop collecting data after 140 mm of peel (280 mm of crosshead travel), in accordance with the ASTM method.

The cotton standard fabric and coated film samples were first cut to 3 inch (7.6 cm) wide×10 inch (25.4 cm) long pieces. The long dimension of the piece of coated film was aligned with the manufacturing machine-direction (MD) of the film. The long dimension of the piece of cotton fabric was parallel to the wale of the fabric. The cut pieces were then conditioned for 5 days at 23° C. and 50% relative humidity, and then tested in the same environment.

After conditioning, one piece of the cotton fabric was bonded a corresponding piece of the coated film, one set at a time, to provide laminated sets of fabric and coated film. Bonding was completed by placing the cotton wale side up on a flat, level surface. A 3×3 inch (7.6×7.6 cm) release strip was placed on one end of the long-dimension of the cotton fabric (to prevent bonding along the one end). Then, the coated film was placed with the coating against the cotton fabric, with the edges of the film coincident with the edges of the cotton. A 4.5 lb (2.04 Kg) PSTC rubber-coated hand roller was rolled down the center of the laminated 10 inch (25.4 cm) long sample (with no added pressure) and rolled from one end to the other at a rate of 24 inches (61 cm) per minute, then rolled back to the opposite end at 24 inches (61 cm) per minute to complete the bond. The width of a laminated set was cut and divided into individual specimens having a 1 inch (2.54 cm) width and tested immediately. The cutting of each laminated set was conducted in a JDC Precision cutter supplied by Thwing-Albert, a business having offices located in Philadelphia, Pa., U.S.A.

After cutting, the 3×3 inch (7.6×7.6 cm) release paper strip was removed, and a 1.875×0.375 inches (4.76×0.95 cm) jumbo-sized paper clip was used to clip the laminate at the initial bond length to prevent peeling during the loading the specimen into the grips of testing system. The cotton fabric was held in the lower grip; the film was held in the upper grip. The clip was then removed, and a pencil was used to support the tail of the specimen in a horizontal position. The crosshead was activated and the pencil was used to support the specimen throughout the test. Five specimens per sample code were tested, and the peel test data from five specimens were arithmetically averaged to determine a peel-strength value for the sample. Data from the peel testing are summarized below in Table 4.

TABLE 4
T-Peel (grams), n = 5

| Code | Average (g) | s | Min. | Max. | COV (%) |
|---|---|---|---|---|---|
| PS-578 | 18.6 | 1.9 | 16.0 | 21.2 | 10.24 |
| Q-5432 | 3.0 | 0.6 | 2.1 | 3.7 | 19.44 |
| PS-500 | 1.9 | 0.4 | 1.4 | 2.2 | 19.48 |

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A personal care article having a longitudinal-direction; a relatively shorter, lateral cross-direction; a first end-edge; a longitudinally-opposed second end-edge;
a first side-edge; and a laterally-opposed second side-edge; the article comprising:
a liquid-permeable topsheet layer;
a backsheet layer operatively connected in facing relation with the topsheet layer;
a first end-notch formed to extend inward from the first end-edge by a first end-notch length;
a second end-notch formed to extend inward from the second end-edge by a second end-notch length; and
a first, end-line of bending weakness, which extends from an inboard, apex end region of the first end-notch.

2. A personal care article as recited in claim 1, wherein the article further includes a first side-notch formed to extend inward from the first side-edge by a first side-notch length; and a second side-notch formed to extend inward from the second side-edge by a second side-notch length.

3. A personal care article as recited in claim 1, wherein the first, end-line of bending weakness extends from the inboard, apex end region of the first end-notch, and has a line-length of at least a minimum of about 1 mm.

4. A personal care article as recited in claim 3, wherein the first, end-line of bending weakness includes a first, end-slit, which has a slit-width of not more than a maximum of about 5 mm, and a slit-length of at least a minimum of about 3 mm.

5. A personal care article as recited in claim 3, further comprising a first, end-slit tear-stop located at an inboard, terminal end region of the first end-slit.

6. A personal care article as recited in claim 5, wherein the first, end-slit tear-stop has a tear-strength which is greater than a tear strength of the article at a location that is substantially immediately adjacent the tear-stop.

7. A personal care article as recited in claim 1, further comprising a second, end-line of bending weakness which extends from an inboard, apex end region of the second end-notch.

8. A personal care article as recited in claim 7, wherein the second, end-line of bending weakness has a line-length of at least a minimum of about 1 mm.

9. A personal care article as recited in claim 7, wherein the second, end-line of bending weakness includes a second, end-slit which extends from the inboard, apex end region of the second end-notch.

10. A personal care article as recited in claim 9, wherein the second, end-slit has a slit-width of not more than a maximum of about 2 mm, and a slit-length of at least a minimum of about 3 mm.

11. A personal care article as recited in claim 9, further comprising a second, end-slit tear-stop located at an inboard, terminal end region of the second end-slit.

12. A personal care article as recited in claim 1, further comprising a first, complementary, end-line of bending weakness which extends from the inboard, apex end region of the first end-notch.

13. A personal care article as recited in claim 12, wherein the first, complementary, end-line of bending weakness includes a first, complementary end-slit which extends from the inboard, apex end region of the first end-notch.

14. A personal care article as recited in claim 13, further comprising
a first, complementary, end-slit tear-stop located at an inboard, terminal end region of the first, complementary end-slit.

15. A personal care article as recited in claim 1, further comprising a second, complementary, end-line of bending weakness which extends from the inboard, apex end region of the first end-notch.

16. A personal care article as recited in claim 15, wherein the second, complementary, end-line of bending weakness includes a second, complementary, end-slit which extends from the inboard, apex end region of the second end-notch.

17. A personal care article as recited in claim 16, further comprising
a second, complementary, end-slit tear-stop located at an inboard, terminal end region of the second, complementary end-slit.

18. A personal care article as recited in claim 1, further comprising an absorbent body sandwiched between the topsheet layer and backsheet layer.

19. A personal care article as recited in claim 1, further comprising
a garment-attachment mechanism operatively connected to a garment-side of the backsheet layer.

20. A personal care article as recited in claim 1, further comprising a slip-resistant, garment-engagement mechanism operatively connected, to a garment-side of the backsheet layer; the garment-engagement mechanism is configured to provide a low-peel-strength engagement with a wearer's outer-garment.
